SEQUENCE LISTING (140 sequences)

SEQ ID NO: 1 - protein, 20 aa, Severe Acute Respiratory Syndrome-associated coronavirus (SARS-CoV)
MYSFVSEETGTLIVNSVLLF

SEQ ID NO: 2 - protein, 27 aa, Severe Acute Respiratory Syndrome-associated coronavirus (SARS-CoV)
SLVKPTVYVYSRVKNLNSSEGVPDLLV

SEQ ID NO: 3 - protein, 27 aa, Severe Acute Respiratory Syndrome-associated coronavirus (SARS-CoV)
LTSGSDLDRSTTFDDVQAPNYTQHTSS

SEQ ID NO: 4 - protein, 23 aa, Severe Acute Respiratory Syndrome-associated coronavirus (SARS-CoV)
QHTSSMRGVYYPDEIFRSDTLYL

SEQ ID NO: 5 - protein, 24 aa, Severe Acute Respiratory Syndrome-associated coronavirus (SARS-CoV)
FAATEKSNVVRGWVFGSTMNNKSQ

SEQ ID NO: 6 - protein, 20 aa, Severe Acute Respiratory Syndrome-associated coronavirus (SARS-CoV)
FELSDNPFFAVSKPMGTQTH

SEQ ID NO: 7 - protein, 29 aa, Severe Acute Respiratory Syndrome-associated coronavirus (SARS-CoV)
DAFSLDVSEKSGNFKHLREFVFKNKDGFL

SEQ ID NO: 8 - protein, 21 aa, Severe Acute Respiratory Syndrome-associated coronavirus (SARS-CoV)
GYLKPTTFMLKYDENGTITDA

SEQ ID NO: 9 - protein, 21 aa, Severe Acute Respiratory Syndrome-associated coronavirus (SARS-CoV)
NATKFPSVYAWERKKISNSVA

SEQ ID NO: 10 - protein, 21 aa, Severe Acute Respiratory Syndrome-associated coronavirus (SARS-CoV)
GQTGVIADYNYKLPDDFMGSV

SEQ ID NO: 11 - protein, 25 aa, Severe Acute Respiratory Syndrome-associated coronavirus (SARS-CoV)
VLAWNTRNIDATSTGNYNYKYRYLR

SEQ ID NO: 12 - protein, 33 aa, Severe Acute Respiratory Syndrome-associated coronavirus (SARS-CoV)
GVLTPSSKRFQPFQQFGRDVSDFTDSVRDPKTS

SEQ ID NO: 13 - protein, 21 aa, Severe Acute Respiratory Syndrome-associated coronavirus (SARS-CoV)
YHTVSLLRSTSQKSIVAYTMS

SEQ ID NO: 14 - protein, 28 aa, Severe Acute Respiratory Syndrome-associated coronavirus (SARS-CoV)
SGIAAEQDRNTREVFAQVKQMYKTPTLK

SEQ ID NO: 15 - protein, 29 aa, Severe Acute Respiratory Syndrome-associated coronavirus (SARS-CoV)
KQMYKTPTLKYFGGFNFSQILPDPLKPTK

SEQ ID NO: 16 - protein, 21 aa, Severe Acute Respiratory Syndrome-associated coronavirus (SARS-CoV)
GVTQNVLYENQKQIANQFNKA

SEQ ID NO: 17 - protein, 20 aa, Severe Acute Respiratory Syndrome-associated coronavirus (SARS-CoV)
NQKQIANQFNKAISQIQESL

SEQ ID NO: 18 - protein, 31 aa, Severe Acute Respiratory Syndrome-associated coronavirus (SARS-CoV)
LHVTYVPSQERNFTTAPAISHEGKAYFPREG

SEQ ID NO: 19 - protein, 33 aa, Severe Acute Respiratory Syndrome-associated coronavirus (SARS-CoV)
IGIINNTVYDPLQPELDSFKEELDKYFKNHTSP

SEQ ID NO: 20 - protein, 23 aa, Severe Acute Respiratory Syndrome-associated coronavirus (SARS-CoV)
NIQKEIDRLNEVAKNLNESLIDL

SEQ ID NO: 21 - protein, 30 aa, Severe Acute Respiratory Syndrome-associated coronavirus (SARS-CoV)
NLNESLIDLQELGKYEQYIKWPWYVWLGFI

SEQ ID NO: 22 - protein, 30 aa, Severe Acute Respiratory Syndrome-associated coronavirus (SARS-CoV); identical to SEQ ID NO: 21

SEQ ID NO: 23 - protein, 18 aa, Severe Acute Respiratory Syndrome-associated coronavirus (SARS-CoV)
LLQFAYSNRNRFLYIIKL

SEQ ID NO: 24 - protein, 20 aa, Severe Acute Respiratory Syndrome-associated coronavirus (SARS-CoV)
RLFARTRSMWSFNPETNILL

SEQ ID NO: 25 - protein, 14 aa, Severe Acute Respiratory Syndrome-associated coronavirus (SARS-CoV)
IVTRPLMESELVIG

SEQ ID NO: 26 - protein, 18 aa, Severe Acute Respiratory Syndrome-associated coronavirus (SARS-CoV)
GHSLGRSDIKDLPKEITV

SEQ ID NO: 27 - protein, 22 aa, Severe Acute Respiratory Syndrome-associated coronavirus (SARS-CoV)
AYNRYRIGNYKLNTDHAGSNDN

SEQ ID NO: 28 - protein, 34 aa, Severe Acute Respiratory Syndrome-associated coronavirus (SARS-CoV)
MSDNGPQSNQRSAPRITFGGPTDSTDNNQNGGRN

SEQ ID NO: 29 - protein, 31 aa, Severe Acute Respiratory Syndrome-associated coronavirus (SARS-CoV)
ITFGGPTDSTDNNQNGGRNGARPKQRRPQGL

SEQ ID NO: 30 - protein, 31 aa, Severe Acute Respiratory Syndrome-associated coronavirus (SARS-CoV)
INTNSGPDDQIGYYRRATRRVRGGDGKMKEL

SEQ ID NO: 31 - protein, 28 aa, Severe Acute Respiratory Syndrome-associated coronavirus (SARS-CoV)
ATEGALNTPKDHIGTRNPNNNAATVLQL

SEQ ID NO: 32 - protein, 28 aa, Severe Acute Respiratory Syndrome-associated coronavirus (SARS-CoV)
ASSRSSSRSRGNSRNSTPGSSRGNSPAR

SEQ ID NO: 33 - protein, 38 aa, Severe Acute Respiratory Syndrome-associated coronavirus (SARS-CoV)
GKGQQQQGQTVTKKSAAEASKKPRQKRTATKQYNVTQA

SEQ ID NO: 34 - protein, 19 aa, Severe Acute Respiratory Syndrome-associated coronavirus (SARS-CoV)
SKKPRQKRTATKQYNVTQA

SEQ ID NO: 35 - protein, 27 aa, Severe Acute Respiratory Syndrome-associated coronavirus (SARS-CoV)
FGRRGPEQTQGNFGDQDLIRQGTDYKH

SEQ ID NO: 36 - protein, 13 aa, Severe Acute Respiratory Syndrome-associated coronavirus (SARS-CoV)
KLDDKDPQFKDNV

SEQ ID NO: 37 - protein, 21 aa, Severe Acute Respiratory Syndrome-associated coronavirus (SARS-CoV)
KKDKKKKTDEAQPLPQRQKKQ

SEQ ID NO: 38 - protein, 31 aa, Severe Acute Respiratory Syndrome-associated coronavirus (SARS-CoV)
DEEEEDDAESEEEEIDETSEHEYGTEDDYQG

SEQ ID NO: 39 - protein, 31 aa, Severe Acute Respiratory Syndrome-associated coronavirus (SARS-CoV)
DEEEEDDAECEEEEIDETCEHEYGTEDDYQG

SEQ ID NO: 40 - protein, 33 aa, Severe Acute Respiratory Syndrome-associated coronavirus (SARS-CoV)
VEEEEEEDWLDDTTEQSEIEPEPEPTPEEPVNQ

SEQ ID NO: 41 - protein, 16 aa, Severe Acute Respiratory Syndrome-associated coronavirus (SARS-CoV)
NGAMQKESDDYIKLNG

SEQ ID NO: 42 - protein, 23 aa, Severe Acute Respiratory Syndrome-associated coronavirus (SARS-CoV)
APKQEEPPNTEDSKTEEKSVVQK

SEQ ID NO: 43 - protein, 25 aa, Severe Acute Respiratory Syndrome-associated coronavirus (SARS-CoV)
EETRKLMPISMDVRAIMATIQRKYK

SEQ ID NO: 44 - protein, 32 aa, Severe Acute Respiratory Syndrome-associated coronavirus (SARS-CoV)
SSKTSEEHFVETVSLAGSYRDWSYSGQRTELG

SEQ ID NO: 45 - protein, 21 aa, Severe Acute Respiratory Syndrome-associated coronavirus (SARS-CoV)
TKKWKFPQVGGLTSIKWADNN

SEQ ID NO: 46 - protein, 23 aa, Severe Acute Respiratory Syndrome-associated coronavirus (SARS-CoV)
EIEPKLDGYYKKDNAYYTEQPID

SEQ ID NO: 47 - protein, 20 aa, Severe Acute Respiratory Syndrome-associated coronavirus (SARS-CoV)
ESQQPTSEEVVENPTIQKEV

SEQ ID NO: 48 - protein, 18 aa, Severe Acute Respiratory Syndrome-associated coronavirus (SARS-CoV)
ENTSITIKKPNELSLALG

SEQ ID NO: 49 - protein, 12 aa, Severe Acute Respiratory Syndrome-associated coronavirus (SARS-CoV)
AKRLAQRVFNNY

SEQ ID NO: 50 - protein, 29 aa, Severe Acute Respiratory Syndrome-associated coronavirus (SARS-CoV)
TFTKSTNSRIRASLPTTIAKNSVKSVAKL

SEQ ID NO: 51 - protein, 11 aa, Severe Acute Respiratory Syndrome-associated coronavirus (SARS-CoV)
NYVKSPKFSKL

SEQ ID NO: 52 - protein, 10 aa, Severe Acute Respiratory Syndrome-associated coronavirus (SARS-CoV)
YKRNRATRVE

SEQ ID NO: 53 - protein, 28 aa, Severe Acute Respiratory Syndrome-associated coronavirus (SARS-CoV)
RDLSLQFKRPINPTDQSSYIVDSVAVKN

SEQ ID NO: 54 - protein, 17 aa, Severe Acute Respiratory Syndrome-associated coronavirus (SARS-CoV)
LYFDKAGQKTYERHPLS

SEQ ID NO: 55 - protein, 15 aa, Severe Acute Respiratory Syndrome-associated coronavirus (SARS-CoV)
VNLDNLRANNTKGSL

SEQ ID NO: 56 - protein, 21 aa, Severe Acute Respiratory Syndrome-associated coronavirus (SARS-CoV)
LSEQLRKQIRSAAKKNNIPFR

SEQ ID NO: 57 - protein, 14 aa, Severe Acute Respiratory Syndrome-associated coronavirus (SARS-CoV)
RGGSYKNDKSSPVV

SEQ ID NO: 58 - protein, 16 aa, Severe Acute Respiratory Syndrome-associated coronavirus (SARS-CoV)
GSISYSELRPDTRYVL

SEQ ID NO: 59 - protein, 18 aa, Severe Acute Respiratory Syndrome-associated coronavirus (SARS-CoV)
LNNEHYRALSGVFSGVDA

SEQ ID NO: 60 - protein, 10 aa, Severe Acute Respiratory Syndrome-associated coronavirus (SARS-CoV)
FFNNYLRKRV

SEQ ID NO: 61 - protein, 42 aa, Severe Acute Respiratory Syndrome-associated coronavirus (SARS-CoV)
DTTSYREAASSHLAKALNDFSNSGADVLYQPPQTSITSAVLQ

SEQ ID NO: 62 - protein, 21 aa, Severe Acute Respiratory Syndrome-associated coronavirus (SARS-CoV)
LNPNYEDLLIRKSNHSFLVQA

SEQ ID NO: 63 - protein, 21 aa, Severe Acute Respiratory Syndrome-associated coronavirus (SARS-CoV)
KVDTSNPKTPKYKFVRIQPGQ

SEQ ID NO: 64 - protein, 21 aa, Severe Acute Respiratory Syndrome-associated coronavirus (SARS-CoV)
NLVAMKYNYEPLTQDHVDILG

SEQ ID NO: 65 - protein, 14 aa, Severe Acute Respiratory Syndrome-associated coronavirus (SARS-CoV)
ADTSLSGYRLKDSV

SEQ ID NO: 66 - protein, 11 aa, Severe Acute Respiratory Syndrome-associated coronavirus (SARS-CoV)
RTVYDDAARRV

SEQ ID NO: 67 - protein, 18 aa, Severe Acute Respiratory Syndrome-associated coronavirus (SARS-CoV)
DYLVSTQEFRYMNSQGLL

SEQ ID NO: 68 - protein, 14 aa, Severe Acute Respiratory Syndrome-associated coronavirus (SARS-CoV)
QQLRVESSSKLWAQ

SEQ ID NO: 69 - protein, 9 aa, Severe Acute Respiratory Syndrome-associated coronavirus (SARS-CoV)
KDTTEAFEK

SEQ ID NO: 70 - protein, 17 aa, Severe Acute Respiratory Syndrome-associated coronavirus (SARS-CoV)
DINRLSEEMLDNRATLQ

SEQ ID NO: 71 - protein, 26 aa, Severe Acute Respiratory Syndrome-associated coronavirus (SARS-CoV)
YATAQEAYEQAVANGDSEVVLKKLKK

SEQ ID NO: 72 - protein, 17 aa, Severe Acute Respiratory Syndrome-associated coronavirus (SARS-CoV)
AKSEFDRDAAMQRKLEK

SEQ ID NO: 73 - protein, 16 aa, Severe Acute Respiratory Syndrome-associated coronavirus (SARS-CoV)
YKQARSEDKRAKVTSA

SEQ ID NO: 74 - protein, 19 aa, Severe Acute Respiratory Syndrome-associated coronavirus (SARS-CoV)
LRKLDNDALNNIINNARDG

SEQ ID NO: 75 - protein, 17 aa, Severe Acute Respiratory Syndrome-associated coronavirus (SARS-CoV)
DYGTYKNTSDGNTFTYA

SEQ ID NO: 76 - protein, 26 aa, Severe Acute Respiratory Syndrome-associated coronavirus (SARS-CoV)
EIQQVVDADSKIVQLSEINMDNSPNL

SEQ ID NO: 77 - protein, 11 aa, Severe Acute Respiratory Syndrome-associated coronavirus (SARS-CoV)
NNELSPVALRQ

SEQ ID NO: 78 - protein, 17 aa, Severe Acute Respiratory Syndrome-associated coronavirus (SARS-CoV)
DDNALAYYNNSKGGRFV

SEQ ID NO: 79 - protein, 20 aa, Severe Acute Respiratory Syndrome-associated coronavirus (SARS-CoV)
LSDHQDLKWARFPKSDGTGT

SEQ ID NO: 80 - protein, 24 aa, Severe Acute Respiratory Syndrome-associated coronavirus (SARS-CoV)
YTELEPPSRFVTDTPKGPKVKYLY

SEQ ID NO: 81 - protein, 23 aa, Severe Acute Respiratory Syndrome-associated coronavirus (SARS-CoV)
AFAVDPAKAYKDYLASGGQPITN

SEQ ID NO: 82 - protein, 17 aa, Severe Acute Respiratory Syndrome-associated coronavirus (SARS-CoV)
TVTPEANMDQESFGGAS

SEQ ID NO: 83 - protein, 20 aa, Severe Acute Respiratory Syndrome-associated coronavirus (SARS-CoV)
RSHIDHPNPKGFSDLKGKYV

SEQ ID NO: 84 - protein, 20 aa, Severe Acute Respiratory Syndrome-associated coronavirus (SARS-CoV)
RCHIDHPNPKGFCDLKGKYV

SEQ ID NO: 85 - protein, 9 aa, Severe Acute Respiratory Syndrome-associated coronavirus (SARS-CoV)
DQLREPLMQ

SEQ ID NO: 86 - protein, 20 aa, Severe Acute Respiratory Syndrome-associated coronavirus (SARS-CoV)
KTNSSRFQEKDEEGNLLDSY

SEQ ID NO: 87 - protein, 22 aa, Severe Acute Respiratory Syndrome-associated coronavirus (SARS-CoV)
VVKRHTMSNYQHEETIYNLVKD

SEQ ID NO: 88 - protein, 13 aa, Severe Acute Respiratory Syndrome-associated coronavirus (SARS-CoV)
VPHISRQRLTKYT

SEQ ID NO: 89 - protein, 25 aa, Severe Acute Respiratory Syndrome-associated coronavirus (SARS-CoV)
DDDYFNKKDWYDFVENPDILRVYAN

SEQ ID NO: 90 - protein, 20 aa, Severe Acute Respiratory Syndrome-associated coronavirus (SARS-CoV)
TLDNQDLNGNWYDFGDFVQV

SEQ ID NO: 91 - protein, 26 aa, Severe Acute Respiratory Syndrome-associated coronavirus (SARS-CoV)
KYDFTEERLSLFDRYFKYWDQTYHPN

SEQ ID NO: 92 - protein, 25 aa, Severe Acute Respiratory Syndrome-associated coronavirus (SARS-CoV)
NNVAFQTVKPGNFNKDFYDFAVSKG

SEQ ID NO: 93 - protein, 18 aa, Severe Acute Respiratory Syndrome-associated coronavirus (SARS-CoV)
DGNAAISDYDYYRYNLPT

SEQ ID NO: 94 - protein, 27 aa, Severe Acute Respiratory Syndrome-associated coronavirus (SARS-CoV)
YYDSMSYEDQDALFAYTKRNVIPTITQ

SEQ ID NO: 95 - protein, 20 aa, Severe Acute Respiratory Syndrome-associated coronavirus (SARS-CoV)
NLKYAISAKNRARTVAGVSI

SEQ ID NO: 96 - protein, 17 aa, Severe Acute Respiratory Syndrome-associated coronavirus (SARS-CoV)
TNRQFHQKLLKSIAATR

SEQ ID NO: 97 - protein, 22 aa, Severe Acute Respiratory Syndrome-associated coronavirus (SARS-CoV)
VLARKHNTSSNLSHRFYRLANE

SEQ ID NO: 98 - protein, 21 aa, Severe Acute Respiratory Syndrome-associated coronavirus (SARS-CoV)
LYRNRDVDHEFVDEFYAYLRK

SEQ ID NO: 99 - protein, 21 aa, Severe Acute Respiratory Syndrome-associated coronavirus (SARS-CoV)
KQGDDYVYLPYPDPSRILGAG

SEQ ID NO: 100 - protein, 26 aa, Severe Acute Respiratory Syndrome-associated coronavirus (SARS-CoV)
DAYPLTKHPNQEYADVFHLYLQYIRK

SEQ ID NO: 101 - protein, 17 aa, Severe Acute Respiratory Syndrome-associated coronavirus (SARS-CoV)
LTNDNTSRYWEPEFYEA

SEQ ID NO: 102 - protein, 21 aa, Severe Acute Respiratory Syndrome-associated coronavirus (SARS-CoV)
YSKSHKPPISFPLSANGQVFG

SEQ ID NO: 103 - protein, 21 aa, Severe Acute Respiratory Syndrome-associated coronavirus (SARS-CoV)
YCKSHKPPISFPLCANGQVFG

SEQ ID NO: 104 - protein, 20 aa, Severe Acute Respiratory Syndrome-associated coronavirus (SARS-CoV)
KLFAAETLKATEETFKLSYG

SEQ ID NO: 105 - protein, 21 aa, Severe Acute Respiratory Syndrome-associated coronavirus (SARS-CoV)
IATVREVLSDRELHLSWEVGK

SEQ ID NO: 106 - protein, 32 aa, Severe Acute Respiratory Syndrome-associated coronavirus (SARS-CoV)
KPRPPLNRNYVFTGYRVTKNSKVQIGEYTFEK

SEQ ID NO: 107 - protein, 18 aa, Severe Acute Respiratory Syndrome-associated coronavirus (SARS-CoV)
GEYTFEKGDYGDAVVYRG

SEQ ID NO: 108 - protein, 25 aa, Severe Acute Respiratory Syndrome-associated coronavirus (SARS-CoV)
LNISDEFSSNVANYQKVGMQKYSTL

SEQ ID NO: 109 - protein, 22 aa, Severe Acute Respiratory Syndrome-associated coronavirus (SARS-CoV)
DNKLKAHKDKSAQSFKMFYKGV

SEQ ID NO: 110 - protein, 20 aa, Severe Acute Respiratory Syndrome-associated coronavirus (SARS-CoV)
TRNPAWRKAVFISPYNSQNA

SEQ ID NO: 111 - protein, 30 aa, Severe Acute Respiratory Syndrome-associated coronavirus (SARS-CoV)
QTVDSSQGSEYDYVIFTQTTETAHSSNVNR

SEQ ID NO: 112 - protein, 25 aa, Severe Acute Respiratory Syndrome-associated coronavirus (SARS-CoV)
SDRDLYDKLQFTSLEIPRRNVATLQ

SEQ ID NO: 113 - protein, 16 aa, Severe Acute Respiratory Syndrome-associated coronavirus (SARS-CoV)
DIPGIPKDMTYRRLIS

SEQ ID NO: 114 - protein, 23 aa, Severe Acute Respiratory Syndrome-associated coronavirus (SARS-CoV)
GYVDTENNTEFTRVNAKPPPGDQ

SEQ ID NO: 115 - protein, 10 aa, Severe Acute Respiratory Syndrome-associated coronavirus (SARS-CoV)
GNLQSNHDQH

SEQ ID NO: 116 - protein, 20 aa, Severe Acute Respiratory Syndrome-associated coronavirus (SARS-CoV)
YDAQPSSDKAYKIEELFYSY

SEQ ID NO: 117 - protein, 24 aa, Severe Acute Respiratory Syndrome-associated coronavirus (SARS-CoV)
DKFTDGVSLFWNSNVDRYPANAIV

SEQ ID NO: 118 - protein, 24 aa, Severe Acute Respiratory Syndrome-associated coronavirus (SARS-CoV)
DKFTDGVCLFWNCNVDRYPANAIV

SEQ ID NO: 119 - protein, 20 aa, Severe Acute Respiratory Syndrome-associated coronavirus (SARS-CoV)
YYSDSPSESHGKQVVSDIDY

SEQ ID NO: 120 - protein, 22 aa, Severe Acute Respiratory Syndrome-associated coronavirus (SARS-CoV)
NLGGAVSRHHANEYRQYLDAYN

SEQ ID NO: 121 - protein, 13 aa, Severe Acute Respiratory Syndrome-associated coronavirus (SARS-CoV)
IYKQFDTYNLWNT

SEQ ID NO: 122 - protein, 14 aa, Severe Acute Respiratory Syndrome-associated coronavirus (SARS-CoV)
DVEIFENKTTLPVN

SEQ ID NO: 123 - protein, 19 aa, Severe Acute Respiratory Syndrome-associated coronavirus (SARS-CoV)
ELWAKRNIKPVPEIKLNNL

SEQ ID NO: 124 - protein, 20 aa, Severe Acute Respiratory Syndrome-associated coronavirus (SARS-CoV)
NTVIWDYKREAPAHVSTIGV

SEQ ID NO: 125 - protein, 16 aa, Severe Acute Respiratory Syndrome-associated coronavirus (SARS-CoV)
TDIAKKPTESASSSLT

SEQ ID NO: 126 - protein, 44 aa, Severe Acute Respiratory Syndrome-associated coronavirus (SARS-CoV)
VLFDGRVEGQVDLFRNARNGVLITEGSVKGLTPSKGPAQASVNG

SEQ ID NO: 127 - protein, 19 aa, Severe Acute Respiratory Syndrome-associated coronavirus (SARS-CoV)
TLIGESVKTQFNYFKKVDG

SEQ ID NO: 128 - protein, 22 aa, Severe Acute Respiratory Syndrome-associated coronavirus (SARS-CoV)
ETYFTQSRDLEDFKPRSQMETD

SEQ ID NO: 129 - protein, 13 aa, Severe Acute Respiratory Syndrome-associated coronavirus (SARS-CoV)
DEFIQRYKLEGYA

SEQ ID NO: 130 - protein, 35 aa, Severe Acute Respiratory Syndrome-associated coronavirus (SARS-CoV)
AKRSQDSPLKLEDFIPMDSTVKNYFITDAQTGSSK

SEQ ID NO: 131 - protein, 16 aa, Severe Acute Respiratory Syndrome-associated coronavirus (SARS-CoV)
LWSKDGHVETFYPKLQ

SEQ ID NO: 132 - protein, 24 aa, Severe Acute Respiratory Syndrome-associated coronavirus (SARS-CoV)
NLYKMQRMLLEKSDLQNYGENAVI

SEQ ID NO: 133 - protein, 22 aa, Severe Acute Respiratory Syndrome-associated coronavirus (SARS-CoV)
YDPRTKHVTKENDSKEGFFTYL

SEQ ID NO: 134 - protein, 35 aa, Severe Acute Respiratory Syndrome-associated coronavirus (SARS-CoV)
NYLGKPKEQIDGYTMHANYIFWRNTNPIQLSSYSL

SEQ ID NO: 135 - protein, 30 aa, Severe Acute Respiratory Syndrome-associated coronavirus (SARS-CoV)
SLKENQINDMIYSLLEKGRLIIRENNRVVV

SEQ ID NO: 136 - protein, 32 aa, Severe Acute Respiratory Syndrome-associated coronavirus (SARS-CoV)
IGIINNTVYDPLQPELDSFKEELDKYFKNHTS

SEQ ID NO: 137 - protein, 33 aa, Severe Acute Respiratory Syndrome-associated coronavirus (SARS-CoV)
SDNGPQSNQRSAPRITFGGPTDSTDNNQNGGRN

SEQ ID NO: 138 - protein, 41 aa, Severe Acute Respiratory Syndrome-associated coronavirus (SARS-CoV)
AYKTFPPTEPKKDKKKKTDEAQPLPQRQKKQPTVTLLPAAD

SEQ ID NO: 139 - protein, 41 aa, Severe Acute Respiratory Syndrome-associated coronavirus (SARS-CoV)
SDNGPQSNQRSAPRITFGGPTDSTDNNQNGGRNGARPKQRR

SEQ ID NO: 140 - protein, 41 aa, Severe Acute Respiratory Syndrome-associated coronavirus (SARS-CoV)
SDVVIGIINNTVYDPLQPELDSFKEELDKYFKNHTSPDVDL

(19) United States
(12) Patent Application Publication
(10) Pub. No.: US 2006/0263765 A1
(43) Pub. Date: Nov. 23, 2006

(54) Peptides and mixtures thereof for use in the detection of severe acute respiratory syndrome-associated coronavirus (sars)

(76) Inventors: Michel Houde, Coteau-du-Lac (CA); Jena-Michel Lacroix, Montreal (CA)

Correspondence Address: CHOATE, HALL & STEWART LLP, TWO INTERNATIONAL PLACE, BOSTON, MA, 02110, US

(21) Appl. No.: 10/556,204

(22) PCT Filed: May 5, 2004
(86) PCT No.: PCT/CA04/00672

§ 371 Date: Mar. 9, 2006

(30) Foreign Application Priority Data
May 9, 2003 (CA) 2,428,443
Sep. 23, 2003 (CA) 2,441,677

(51) Int. Cl. C12Q 1/70; C07K 14/165; C07K 16/10

(52) U.S. Cl. 435/5; 530/350; 530/388.3

(57) ABSTRACT

The present invention relates to novel peptides and mixtures thereof useful for detecting Severe Acute Respiratory Syndrome-associated coronavirus (SARS-CoV) infections in humans and animals. Therefore, the present invention provides SARS-CoV diagnostic methods and kits.

### FIELD OF THE INVENTION

The present invention relates to novel peptides and mixtures thereof useful for detecting Severe Acute Respiratory Syndrome-associated coronavirus (SARS-CoV) infections in humans and animals.

### BACKGROUND OF THE INVENTION

SARS, an atypical pneumonia of unknown etiology, was recognized at the end of February 2003 by the World Health Organization. In April 2003, scientists around the world demonstrated that a previously unrecognized coronavirus (SARS-CoV) was probably the cause of SARS (Drosten et al., 2003; Ksiazek et al., 2003; Peiris et al., 2003).

Few serological diagnostic tests for SARS-CoV have been developed so far (Drosten et al., 2003; Ksiazek et al., 2003; Peiris et al, 2003). Indirect immunofluorescence assay (IFA) were first introduced (Drosten et al., 2003; Ksiazek et al., 2003; Peiris et al., 2003) soon followed by ELISA (Ksiazek et al., 2003; Peiris et al., 2003). The IFAs were all based on the fixation of SARS-infected cells, the subsequent binding of human anti-SARS antibodies and their labeling with a fluorescent anti-human antibody. The first ELISA were based on the use of whole virus lysates (WVL), namely a preparation of virus enriched from tissue culture. However, as it has been experienced with other viruses, the lack of purity of whole viral lysates usually causes higher background levels in the assay, as antibodies directed against contaminants of the whole viral lysate will also be captured. This lack of purity decreases the sensitivity of the assay and a lot of patients showing low anti-SARS-CoV antibody levels will not be detected. For the same reasons, the specificity of these whole viral lysate assays is also unacceptably low. Patients harboring high antibody levels directed against the contaminants of the whole viral lysate preparation will be detected as SARS-positive cases. As a matter of fact, since the first application of the present document, a number of publications have reported the usefulness of synthetic peptides and recombinant proteins for the specific detection of anti-SARS-CoV antibodies (Ho et al., 2004; Wang et al.,2003; Wu et al.,2004).

Thus, there is a definite need to develop a more sensitive and more specific test for the diagnosis of SARS-CoV infections.

### SUMMARY OF THE INVENTION

The present invention concerns specific SARS-CoV peptides and mixtures thereof for the development of a SARS-CoV diagnostic methods and kits.

More precisely, an object of the present invention is to provide an isolated peptide comprising an amino acid sequence selected from the group consisting of SEQ ID NOS: 1 to 4, 6 to 16, 18 to 22, 24 to 33, 35 to 41, 43 to 62, 64 to 73, 75 to 77, 79 to 81, 83 to 100, 102 to 105, 107 to 113, 115 to 118, 120 to 127 and 129 to 140 and analogues thereof.

Another object of the present invention is to provide an isolated peptide having the formula a-X-c-Z-b wherein: X and Z has an amino acid sequence which is 85% identical to an amino acid sequence independently selected from the group consisting of SEQ ID NOS: 1 to 4, 6 to 16, 18 to 22, 24 to 33, 35 to 41, 43'to 62, 64 to 73, 75 to 77, 79 to 81, 83 to 100, 102 to 105, 107 to 113, 115 to 118, 120 to 127 and 129 to 140 and analogues thereof, and wherein:
a is an amino terminus, one to eight amino acids or a substituent effective to facilitate coupling or to improve the immunogenic or antigenic activity of the peptide or to facilitate attachment to a support matrix;
b is a carboxy terminus, one to eight amino acids or a substituent effective to facilitate coupling or to improve the immunogenic or antigenic activity of the peptide or to facilitate attachment to the support matrix; and
c is a linker of one or two amino acids or a substituent effective to facilitate coupling of the two peptides in tandem or to improve the immunogenic or antigenic activity of the tandem peptide or to facilitate attachment to the support matrix.

Another object of the invention concerns a mixture comprising at least two peptides or analogues thereof as defined above.

A further object concerns an antibody that specifically binds to a peptide or analogue thereof of the invention or a mixture of antibodies that specifically binds to a peptide or a mixture of antibodies that specifically binds to a mixture of peptides as defined above.

Yet, another object of the invention is to provide an in vitro diagnostic method for the detection of the presence or absence of antibodies indicative of SARS-CoV, which bind with a peptide or analogue thereof according to the invention to form an immune complex, comprising the steps of:
a) contacting the peptide or analogue thereof according to the invention with a biological sample for a time and under conditions sufficient to form an immune complex; and
b) detecting the presence or absence of the immune complex formed in a).

Yet, a further object of the invention is to provide a diagnostic kit for the detection of the presence or absence of antibodies indicative of SARS-CoV, comprising:
a peptide or analogue thereof according to the invention; and
a reagent to detect a peptide-antibody immune complex;
   wherein said peptide or analogue thereof and reagent are present in an amount sufficient to perform said detection.

Another object of the invention is to provide an in vitro diagnostic method for the detection of the presence or absence of peptides or proteins indicative of SARS-CoV, which bind with an antibody according to the invention to form an immune complex, comprising the steps of:
a) contacting the antibody according to the invention with a biological sample for a time and under conditions sufficient to form an immune complex; and
b) detecting the presence or absence of the immune complex formed in a).

A further object of the present invention is to provide a diagnostic kit for the detection of the presence or absence of peptides or proteins indicative of SARS-CoV, comprising:
an antibody according to the invention and
a reagent to detect a peptide-antibody immune complex;
   wherein said antibody and reagent are present in an amount sufficient to perform said detection.

The peptides and mixtures thereof of the present invention are useful for the screening of blood and body fluids for SARS-CoV infection. For example, the peptides described therein and mixtures thereof are useful in a wide variety of specific binding assays for the detection of antibodies to SARS-COV and as immunogens for eliciting antibodies useful for the detection of SARS-CoV antigens.

### BRIEF DESCRIPTION OF THE DRAWINGS

FIG. 1 depicts the Kyle-Doolittle (hydrophilicity plot), Jameson-Wolf (antigenic index) and Emini (surface probability) profiles of the putative Nucleocapsid (N) protein of SARS-CoV, based on the sequence provided by BCCA Genome Sciences Center on Apr. 13, 2003 (Protein ID NP_{—}828858.1; 422 AA).

FIG. 2 shows the amino acids sequence of the peptides contemplated by the present invention, and identified as SEQ ID NOS: 1 to 140.

### DETAILED DESCRIPTION OF THE INVENTION

The present invention provides novel peptides and analogues thereof corresponding to immunodominant regions of the putative spike (S), nucleocapsid (N) and matrix (M) gene products of SARS-CoV. The present invention also provides mixtures and chemical combinations (tandem s) of these peptides and analogues. As will be explain from the following description, these peptides, analogues, mixtures and tandems are useful in a wide variety of diagnostic methods and kits, with respect to SARS-CoV and the infections caused by it.

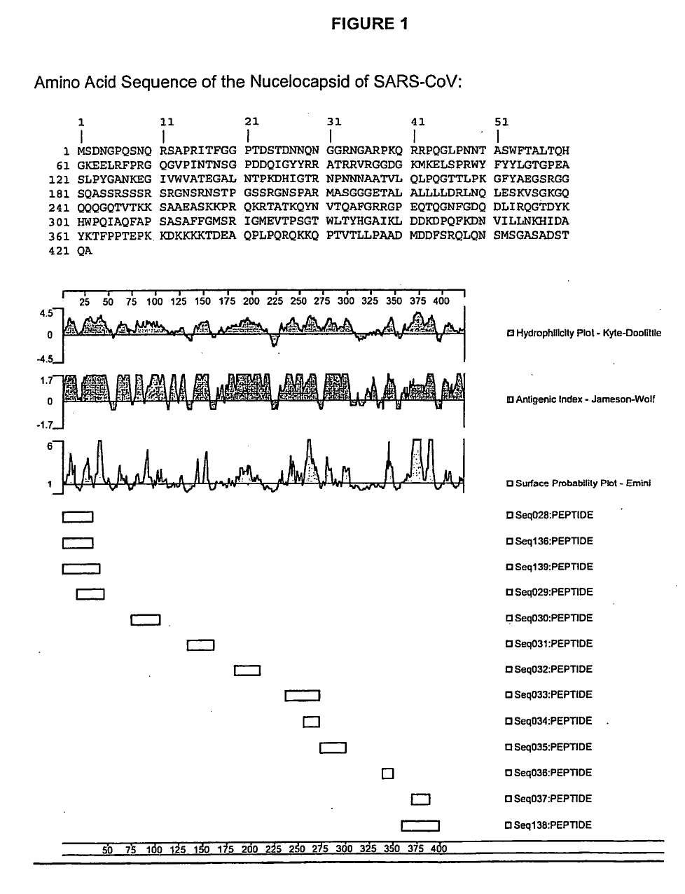

The peptides of the invention are preferably selected on the basis of the analysis of . SARS-CoV proteins with three (3) algorithms for prediction of hydrophilicity plots (Kyle-Doolittle), surface probability plots (Emini) and antigenic indexes (Jameson-Wolf), as shown in FIG. 1. Regions of amino acids showing a positive index for these 3 parameters have a good probability of being immunogenic and consequently forming a linear epitope that can be used for the detection of anti-SARS-CoV antibodies. The following SARS-CoV proteins are analysed: S (Spike; NP_{—}828851.1), N (Nucleocapsid, NP_{—}828858.1), M (Matrix; NP_{—}828855.1), E (small Envelope; NP_{—}828854.1), NSP1 (Non-Structural Protein 1; NP_{—}828862.1) NSP2 (NP_{—}828863.1), NSP3 (NP_{—}828864.1), NSP4 (NP_{—}828865.1), NSP5 (NP-828866.1), NSP6 (NP_{—}828867.1), NSP7 (NP_{—}828868.1), NSP9 (NP_{—}828869.1), NSP10 (NP_{—}828870.1), NSP11 (NP_{—}828871.1), NSP12 (NP_{—}828872.1) and NSP13 (NP_{—}828873.2).

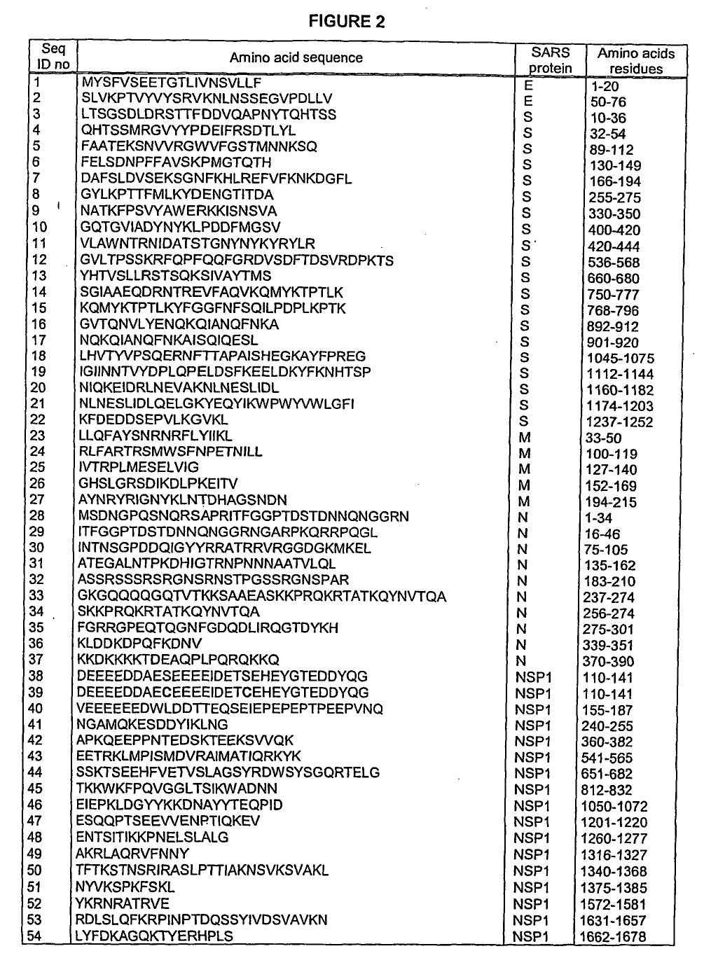

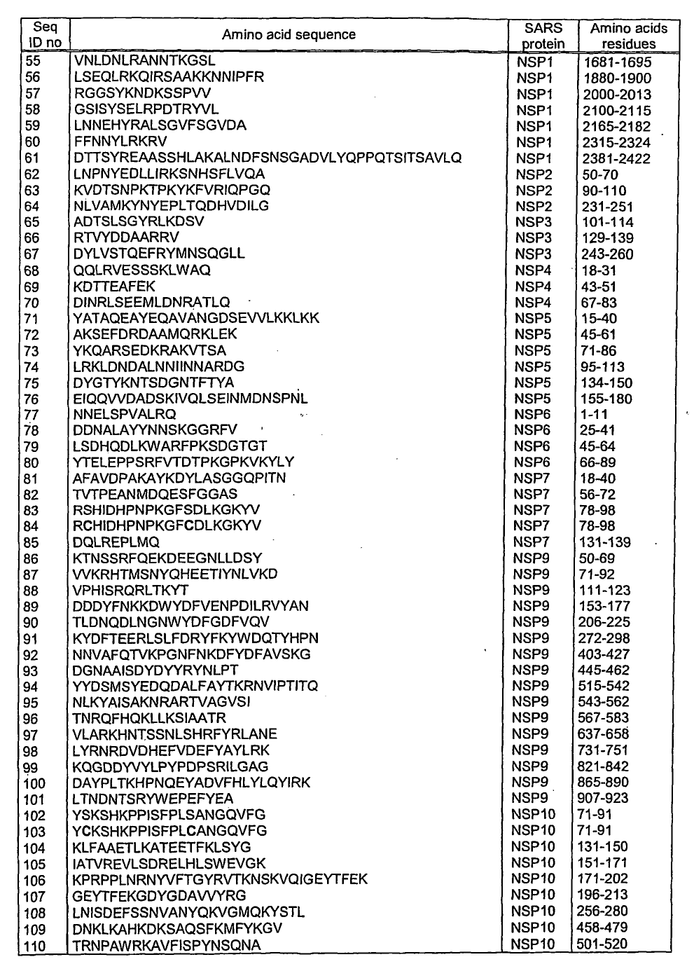

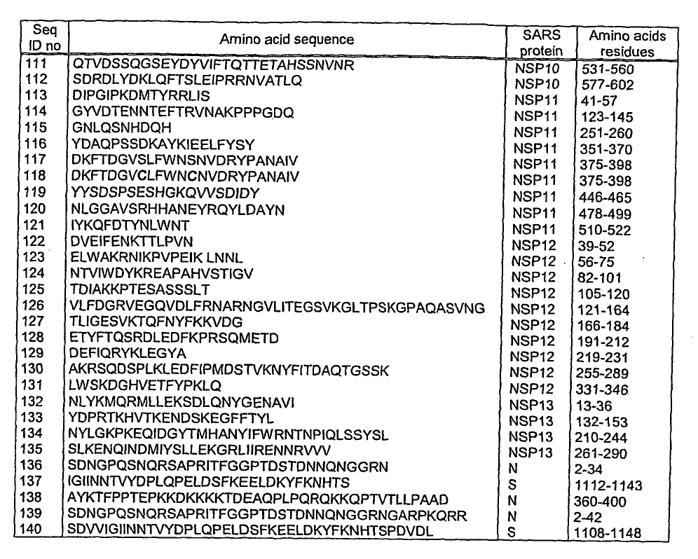

As set forth above, the peptides shown in FIG. 2 were selected for further experimentations. The cysteine residue in positions 19 of Seq ID no:3, 133 of Seq ID no:6, 348 of Seq ID no:9, 419 of Seq ID no:10, 1064 of Seq ID no:18, 158 of Seq ID no:26, 119 and 128 of Seq ID no:38, 550 of Seq ID no:43, 2010 of Seq ID no:57, 2178 of Seq ID no:59, 2390 and 2391 of Seq ID no:61, 113 of Seq ID no:65, 72 of Seq ID no:70, 142 of Seq ID no:75, 73 of Seq ID no:80, 79 and 91 of Seq ID no:84, 53 and 54 of Seq ID no:86 281 of Seq ID no:91, 645 and 646 of Seq ID no:97, 72 and 84 of Seq ID no:102, 471 of Seq ID no:109, 556 of Seq ID no:111, 356 of Seq ID no:116, 382 and 387 of Seq ID no:117, 452 of Seq ID no:119, 484 of Seq ID no:120, 116 of Seq ID no:125, 333 of Seq ID no:131 and 25 of Seq ID no:132 were replaced by a serine residue. With regards to Seq ID no:039, 84, 103 and 118, the original Cys residues were conserved in order to allow the formation of a disulfide bridge.

Peptides of the Invention

According to a first object, the present invention relates an isolated peptide comprising an amino acid sequence selected from the group consisting of SEQ ID NOS: 1 to 4, 6 to 16, 18 to 22, 24 to 33, 35 to 41, 43 to 62, 64 to 73, 75 to 77, 79 to 81, 83 to 100, 102 to 105, 107 to 113, 115 to 118, 120 to 127 and 129 to 140 and analogues thereof.

According to another object, the present invention relates to tandem peptides. Indeed, the present invention relates to an isolated peptide having the formula a-X-c-Z-b wherein: X and Z has an amino acid sequence independently selected from the group consisting of SEQ ID NOS: 1 to 4, 6 to 16, 18 to 22, 24 to 33, 35 to 41, 43 to 62, 64 to 73, 75 to 77, 79 to 81, 83 to 100, 102 to 105, 107 to 113, 115 to 118, 120 to 127 and 129 to 140 and analogues thereof, and wherein:
a is an amino terminus, one to eight amino acids or a substituent effective to facilitate coupling or to improve the immunogenic or antigenic activity of the peptide or to facilitate attachment to a support matrix;
b is a carboxy terminus, one to eight amino acids or a substituent effective to facilitate coupling or to improve the immunogenic or antigenic activity of the peptide or to facilitate attachment to the support matrix; and
c is a linker of one or two amino acids or a substituent effective to facilitate coupling of the two peptides in tandem or to improve the immunogenic or antigenic activity of the tandem peptide or to facilitate attachment to the support matrix.

According to another object of the invention, the present invention also contemplates to provide a mixture comprising at least two peptides or analogues thereof as defined above.

Preferably, the amino acid sequence is selected from the group of amino acid sequences consisting of SEQ ID NOS: 3, 19, 22, 28, 31, 37, 136, 137, 138, 139 and 140 and analogues thereof. Most preferably, the amino acid sequence consists of either SEQ ID NO: 3, 19, 22, 28, 31, 37, 136, 137, 138, 139 or 140 or analogues thereof.

As used herein, “analogues” refer to an amino acid sequence which is at least 85% identical to the entire length of the amino acid sequence of a peptide as defined above. More specifically, the term “analogues” denote amino acid insertions, deletions, substitutions and modifications at one or more sites in the peptide chain in that portion of it that consists of the block of the naturally occurring SARS-CoV amino acid sequences.

Preferred modifications and substitutions to the native amino acid sequence of the peptides of this invention are conservative ones (i.e., those having minimal influence on the secondary structure and hydropathic nature of the peptide). These include substitutions such as those described by Dayhoff in the Atlas of Protein Sequence and Structure 5, 1978 and by Argos in EMBO J., 8, 779-785, 1989. For example, amino acids belonging to one of the following groups represent conservative changes: Ala, Pro, Gly, Glu, Asp, Gln, Asn, Ser, Thr; Cys, Ser, Tyr, Thr; Val, lie, Leu, Met, Ala, Phe; Lys, Arg, His; and Phe, Tyr, Trp, His.

In like manner, methionine (Met), an amino acid which is prone to oxidation, may be replaced in the peptides of this invention by norleucine. The preferred substitutions also include substitutions of D-isomers for the corresponding L-amino acids.

The term “amino acid” as employed in this description (e.g., in the definition of a and b and analogues) except when referring to the native amino acid sequence of the gene products of SARS-CoV, encompasses all of the natural amino acids, those amino acids in their D-configurations, and the known non-native, synthetic, and modified amino acids, such as homocysteine, ornithine, norleucine and β-valine.

As set forth briefly above, it is often useful and certainly within the scope of this invention to modify the peptides of this invention in order to make the chosen peptide more useful as an immunodiagnostic reagent. Such changes, for example, include:
addition of a cysteine residue to one or both terminals in order to facilitate coupling of the peptide to a suitable carrier with heterobi-functional cross-linking reagents, such as sulfosuccinimidyl-4-(p-maleimidophenyl) butyrate. Preferred reagents for effecting such linkages are sulfosuccinimidyl-sulfosuccinimidyl-4-(N-maleimidomethyl)cyclohexane-1-carboxylate and N-succinimidyl-3-(2-pyridyldithio) propionate;
addition of 1 to 8 additional amino acids at one or both terminals of the peptide to facilitate linking of the peptides to each other, for coupling to a support or larger peptide or protein or for modifying the physical or chemical properties of the peptide. Examples of such changes are the addition of N- or C-terminal tyrosine, glutamic acid or aspartic acid as linkers via an esterification reaction and lysine which can be linked via Schiff base or amide formation. As described above, such additional amino acids may include any of the natural amino acids, those amino acids in their D-configurations and the known non-native, synthetic and modified amino acids; and
derivatization of one or both terminals of the peptide by, for example, acylation or amidation. These modifications result in changes in the net charge on the peptide and can also facilitate covalent linking of the peptide to a support matrix, a carrier or another peptide. Examples of the substituents effective to facilitate coupling or to improve the immunogenicity or antigenic activity of the peptide or to facilitate attachment to the support matrix are C₂-C₁₆ acyl groups, polyethylene glycol, phospholipids, human serum albumin (HSA) and polylysine (PLL).

As reflected above, it is within the scope of the invention to employ tandem peptides. These peptides may be homopolymers or copolymers. Physical mixtures of the peptides and tandem peptides of this invention are also within its scope.

To prepare the novel peptides of the invention any of the conventional peptide production methodologies may be used. These include synthesis, recombinant DNA technology and combinations thereof. According to the present invention, solid phase synthesis is preferred. In that synthetic approach, the resin support may be any suitable resin conventionally employed in the art for the solid phase preparation of peptides. Preferably, it is a p-benzyloxy-alcohol polystyrene or p-methylbenzyhydrylamine resin. Following the coupling of the first protected amino acid to the resin support, the amino protecting group is removed by standard methods conventionally employed in the art. After removal of the amino protecting group, the remaining protected amino acids and, if necessary, side chain protected amino acids are coupled, sequentially, in the desired order to obtain the chosen peptide. Alternatively, multiple amino acid groups may be coupled using solution methodology prior to coupling with the resin-supported, amino acid sequence.

The selection of an appropriate coupling reagent follows established art. For instance, suitable coupling reagents are N,N′-diisopropylcarbodiimide or N,N′-dicyclohexylcarbodiimide (DCC) or preferably, benzotriazol-1-yloxy-tris (dimethylamino) phosphonium hexafluoro-phosphate either alone more or preferably in the presence of 1-hydroxybenzotriazole. Another useful coupling procedure employs pre-formed symmetrical anhydrides of protected amino acids.

The necessary α-amino protecting group employed for each amino acid introduced onto the growing polypeptide chain is preferably 9-fluorenylmethyloxycarbonyl (FMOC), although any other suitable protecting group may be employed as long as it does not degrade under the coupling conditions and is readily and selectively removable in the presence of any other protecting group already present in the growing peptide chain.

The criteria for selecting protecting groups for the side chain amino acids are: (a) stability of the protecting group to the various reagents under reaction conditions selective for the removal of the .alpha.-amino protecting group at each step of the synthesis; (b) retention of the protecting group's strategic properties (i.e., not be split off under coupling conditions) and (c) removability of protecting group easily upon conclusion of the peptide synthesis and under conditions that do not otherwise affect the peptide structure.

The fully protected resin-supported peptides are preferably cleaved from the p-benzyloxy alcohol resin with 50% to 60% solution of trifluoroacetic acid in methylene chloride for 1 to 6 hours at room temperature in the presence of appropriate scavengers such as anisole, thioanisole, ethyl methyl sulfide, 1,2-ethanedithiol and related reagents. Simultaneously, most acid labile side chain protecting groups are removed. More acid resistant protecting groups are typically removed by HF treatment.

Methods of Use

The peptides of the present invention are useful as diagnostic reagents for the detection and quantification of SARS-CoV associated antibodies in accordance with methods well-known in the art. These include ELISA, Western blot, fluorescence assay, chemiluminescent assay, radioimmunoassay hemagglutination, turbidimetric assay, immunochromatographic (rapid test), single-dot and multi-dot assay methods. Novel methods such as peptide or protein microarrays or using biosensor labels based on piezoelectricity, surface plasmonon resonance (SPR) or cantilever can also be used.

A preferred convenient and classical technique for the determination of antibodies against SARS-CoV using a peptide or a peptide mixture of this invention is an enzyme-linked immunosorbent assay (ELISA). In this assay, for example, a peptide or mixture of this invention is adsorbed onto, or covalently coupled to, the wells of a microtiter plate. The wells are then treated with the sera or biological fluid to be tested. After washing, anti-human IgG or anti-human IgM or anti-human IgA labeled with peroxidase is added to the wells. The determination of the peroxidase is performed with a corresponding substrate, e.g., 3,3′,5,5′-tetramethylbenzidine.

Without departing from the usefulness of this illustrative assay, the peroxidase can be exchanged by another label, e.g., by a radioactive, fluorescence, chemiluminescence or infra-red emitting label.

Another method for the determination of the presence of antibodies against SARS-CoV in a test sample or sera with the peptides and mixtures of this invention is an enzyme immunological test according to the so-called “Double-Antigen-Sandwich-Assay”. This method is based on the work of Maiolini, as described in Immunological Methods, 20, 25-34, 1978. According to this method, the serum or other analyte to be tested is contacted with a solid phase on which a peptide of this invention has been coated (capture layer) and with a peptide of this invention which has been labeled with peroxidase or other signal (probe layer), using couples of ligands such as biotin-avidin, His₆-Ni-NTA, FITC-anti-FITC or others.

The immunological reaction can be performed in one or two steps. If the immunological reaction is performed in two steps, then a washing step is typically carried out between the two incubations. After the immunological reaction or reactions, a washing step is also usually performed. Thereafter, the peroxidase or other signal is determined, e.g., using o-phenylene diamine for peroxidase. Other enzymes and chromogens, including those already described, can also be employed in this assay.

Suitable support matrices or solid phases for use in the above-described assays and assay methods include but are not limited to, organic and inorganic polymers, e.g., amylases, dextrans, natural or modified celluloses, polyethylene, polystyrene, polyacrylamides, agaroses, magnetite, porous glass powder, polyvinyldiene fluoride (kynar) and latex, the inner wall of test vessels (i.e., test tubes, titer plates or cuvettes of glass or artificial material) as well as the surface of solid bodies (i.e., rods of glass and artificial material, rods with terminal lobes or lamellae). Spheres of glass and artificial material are especially suitable as solid phase carriers.

The peptides of the invention and mixtures thereof are not only useful in the determination and quantification of antibodies against SARS-CoV. They are also useful for the determination and quantification of SARS-CoV antigens themselves because the peptides of the invention, either free, polymerized or conjugated to an appropriate carrier are useful in eliciting antibodies, in particular and preferably monoclonal antibodies, immunologically cross reactive to antigens of SARS-CoV. Such antibodies, for example, can be produced by injecting a mammalian or avian animal with a sufficient amount of the peptide to elicit the desired immune response and recovering said antibodies from the serum of said animals. It is thus another object of the invention to provide an antibody that specifically binds to a peptide or analogue thereof, or to a mixture of peptides according to the invention.

With respect to antibodies of the invention, the term “specifically binds to” refers to antibodies that bind with a relatively high affinity to one or more epitopes of a protein of interest, such as a peptide of the invention, but which do not substantially recognize and bind molecules other than the one(s) of interest. As used herein, the term “relatively high affinity” means a binding affinity between the antibody and the peptide or protein of interest of at least 10⁶ M⁻¹, and preferably of at least about 10⁷ M⁻¹ and even more preferably 10⁸ M⁻¹ to 10¹⁰ M⁻¹. Determination of such affinity is preferably conducted under standard competitive binding immunoassay conditions which is common knowledge to one skilled in the art.

Suitable host animals for eliciting antibodies include, for example, rabbits, horses, goats, guinea pigs, rats, mice, cows, sheep and hens. Preferably, hybridomas producing the desired monoclonal antibodies are prepared using the peptides of this invention and conventional techniques.

For example, the well-known Kohler and Milstein technique for producing monoclonal antibodies may be used. In order to distinguish monoclonal antibodies which are directed against the same antigen, but against different epitopes, the method of Stahil et al. (J. of Immunological Methods, 32, 297-304, 1980) can be used.

Various methods which are generally known can be employed in the determination or quantification of SARS-CoV or a portion thereof using the above antibodies. In one such procedure, known amounts of a serum sample or any other biological fluid to be assayed, a radiolabeled peptide or mixture of this invention and an unlabeled peptide or mixture of this invention are mixed together, a given amount of an antibody to a peptide of this invention, preferably a monoclonal antibody, is added and the mixture allowed to stand. The resulting antibody/antigen complex is then separated from the unbound reagents by procedures known in the art such as treatment with ammonium sulphate, polyethylene glycol, a second antibody either in excess or bound to an insoluble support, or dextran-coated charcoal.

The concentration of the labeled peptide is then determined in either the bound or unbound phase and the SARS-CoV antigen content of the sample determined by comparing the level of labeled component to a standard curve in a manner known per se.

Another suitable method for using these antibodies in assays is the “Double-Antibody-Sandwich-Assay”. According to this assay, the sample to be tested is treated with two different antibodies, e.g., raised by immunizing different animals, e.g., sheep and rabbits with a peptide of this invention or a mixture or combination thereof. One of the antibodies is labeled and the other is coated on a solid phase. The preferred solid phase is a plastic bead and the preferred label is horse-radish peroxidase.

Typically in the “Double-Antibody-Sandwich-Assay”, the sample is incubated with the solid phase antibody and the labeled antibody. However, it is also possible to contact the sample first with the solid phase antibody and, then after an optional washing, to contact the sample with the labeled antibody. Preferably, however, the sample is treated together with the solid phase and the labeled antibody. After the immunological reaction(s), the mixture is washed and the label is determined according to procedures known in the art. In the case where peroxidase is used as the label, the determination maybe performed using a substrate, e.g., with o-phenylene diamine or with tetramethylbenzidine. The amount of the labeled component is proportional to the amount of the antigen(s) present in the analyte or serum sample.

Accordingly, in another object, the present invention thus provides an in vitro diagnostic method for the detection of the presence or absence of antibodies indicative of SARS-CoV, which bind with a peptide or analogue thereof according to the invention to form an immune complex, comprising the steps of:
a) contacting the peptide or analogue thereof according to the invention with a biological sample for a time and under conditions sufficient to form an immune complex; and
b) detecting the presence or absence of the immune complex formed in a).

The invention also provides in a further object, an in vitro diagnostic method for the detection of the presence or absence of peptides or proteins indicative of SARS-CoV, which bind with an antibody according to the invention to form an immune complex, comprising the steps of:
a) contacting the antibody according to the invention with a biological sample for a time and under conditions sufficient to form an immune complex; and
b) detecting the presence or absence of the immune complex formed in a).

A “biological sample” encompasses a variety of sample types obtained from an individual (animal or human) and can be used in a diagnostic method of the invention. The definition encompasses blood and other liquid samples of biological origin, solid tissue samples such as a biopsy specimen or tissue cultures or cells derived therefrom, and the progeny thereof.

The definition also includes samples that have been manipulated in any way after their procurement, such as by treatment with reagents, solubilization, or enrichment for certain components, such as proteins or polypeptides. The term “biological sample” encompasses a clinical sample, and also includes cells in culture, cell supernatants, cell lysates, serum, plasma, biological fluid, and tissue samples.

The methods and assays for the determination and quantification of SARS-CoV antigens or antibodies against this virus, as described above, can also be conducted in suitable test kits characterized by a peptide or mixture of this invention, or antibodies against SARS-CoV elicited by those peptides and mixtures. Such kits typically comprise two or more components necessary for performing a diagnostic assay. Components may be compounds, reagents, containers and/or equipment. For example, one container within a kit may contain a monoclonal antibody or fragment thereof that specifically binds to a peptide of the invention. Such antibodies or fragments may be provided attached to a support material known to one skilled in the art. One or more additional containers may enclose elements, such as reagents or buffers, to be used in the assay.

In this connection, in another object, the present invention provides a diagnostic kit for the detection of the presence or absence of antibodies indicative of SARS-CoV, comprising:
a peptide or analogue thereof according to the invention and
a reagent to detect a peptide-antibody immune complex,

wherein said peptide or analogue thereof and reagent are present in an amount sufficient to perform said detection.

In a preferred embodiment, the kit further comprises a biological reference sample lacking antibodies that immunologically bind with said peptide and a comparison sample comprising antibodies which can specifically bind to said peptide or analogue thereof, wherein said biological reference sample and comparison sample are present in an amount sufficient to perform said detection.

Yet, in another object, there is provided a diagnostic kit for the detection of the presence or absence of peptides or proteins indicative of SARS-CoV, comprising:
an antibody according to the invention and
a reagent to detect a peptide-antibody immune complex;
   wherein said antibody and reagent are present in an amount sufficient to perform said detection.

In a preferred embodiment, the kit further comprises a biological reference sample lacking peptides that immunologically bind with said antibody and a comparison sample comprising peptides which can specifically bind to said antibody, wherein said biological reference sample and comparison sample are present in an amount sufficient to perform said detection.

Preferred procedures for the synthesis and utilization of the peptides of the invention are provided below.

Procedure 1

Preparation of Resins Carrying the N-FMOC Protected Amino Acid Residue

The desired N-FMOC protected amino acid residue in a mixture of methylene chloride (CH₂Cl₂) and dimethylformamide (DMF) (4:1) was added to a suspension of p-benzyloxy alcohol resin in CH₂Cl₂ :DMF (4:1) at 0 C. The mixture was stirred manually for a few seconds and then treated with N,N′-dicyclohexyl-carbodiimide (DCC) followed by a catalytic amount of 4-(dimethylamino) pyridine. The mixture was stirred at 0 C. for an additional 30 minutes and then at room temperature overnight. The filtered resin was washed successively with CH₂Cl₂, DMF and isopropanol (3 washes each) and finally, with CH₂Cl₂. The resin was suspended in CH₂Cl₂, chilled in an ice bath and redistilled pyridine was added to the stirred suspension, followed by benzoyl chloride. Stirring was continued at 0 C. for 30 minutes and then at room temperature for 60 minutes. After filtration, the resin was washed successively with CH₂Cl₂, DMF and isopropanol (3 washes each) and finally with petroleum ether (twice) before being dried under high vacuum to a constant weight. Spectrophotometric determination of substitution according to Meienhofer et al. (Int. J. Peptide Protein Res., 13, 35, 1979) indicates the degree of substitution on the resin.

Procedure 2

Coupling of Subsequent Amino Acids

The resin carrying the N-FMOC protected first amino acid residue was placed in a reaction vessel of a Biosearch 9600 Peptide Synthesizer and treated as follows:
1) Washed with DMF (4 times for 20 sec. each)
2) Prewashed with a 30% solution of piperidine in DMF (3 min.)
3) Deprotected with a 30% solution of piperidine in DMF (7 min.)
4) Washed with DMF (8 times for 20 sec. each)
5) Checked for free amino groups—Kaiser Test (must be positive)
6) The peptide resin was then gently shaken for 1 or 2 hrs with 8 equivalents of the desired FMOC-protected amino acid and 1-hydroxybenzotriazole and benzotriazol-1-yloxy-tris(dimethyl-amino) phosphonium hexafluorophosphate all dissolved in dry redistilled DMF containing 16 equivalents of 4-methylmorpholine.
7) Washed with DMF (6 times for 20 sec. each)

After step 7, an aliquot was taken for a ninhydrin test. If the test was negative, the procedure was repeated from step 1 for coupling of the next amino acid. If the test was positive or slightly positive, steps 6 and 7 were repeated.

The above scheme may be used for coupling each of the amino acids of the peptides described in this invention. N-protection with FMOC may also be used with any of the remaining amino acids throughout the synthesis.

Radiolabeled peptides may be prepared by incorporation of a tritiated amino acid using the above coupling protocol.

After the addition of the last amino acid, the N-FMOC of the N-terminal residue is removed by going back to steps 1-7 of the above scheme. The peptide resin is washed with CH₂Cl₂ and dried in vacuo to give the crude protected peptide.

Procedure 3

Deprotection and Cleavage of the Peptides from the Resin

The protected peptide-resin was suspended in a 55% solution of trifluoroacetic acid (TFA) in CH₂Cl₂, containing 2.5% ethanedithiol and 2.5% anisole. The mixture was flushed with N₂ and stirred for 1.5 hours at room temperature. The mixture was filtered and the resin washed with CH₂Cl₂. The resin was treated again with 20% TFA in CH₂Cl₂ for 5 minutes at room temperature. The mixture was filtered and the resin washed with 20% TFA in CH₂Cl₂ and then washed with CH₂Cl₂. The combined filtrates were evaporated in vacuo below 35 C. and the residue washed several times with dry dimethyl ether. The solid was dissolved in 10% aqueous acetic acid and lyophilized to afford the crude product.

The peptides containing Arg and Cys residues are further deprotected by HF treatment at 0 C. for 1 hour in the presence of anisole and dimethylsulfide. The peptides were extracted with 10% aqueous acetic acid, washed with dimethyl ether and lyophilized to afford the crude peptides.

Procedure 4

Purification of Peptides

The crude peptides were purified by preparative HPLC on a Vydac column 2.5×25 mm) of C₁₈ or C₄ reverse phase packing with a gradient of the mobile phase. The effluent was monitored at 220 nm and subsequently by analytical HPLC. Relevant fractions were pooled, evaporated and lyophilized. The identity of the synthetic peptides was verified by analytical reverse phase chromatography and by amino acid analysis.

Procedure 5

Conjugation of Peptides to Bovine Serum Albumin (BSA) or Keyhole Limpet Hemocyanin (KLH)

Peptides were conjugated to BSA or KLH previously derivatized with either sulfosuccinimidyl 4-(p-maleimidophenyl) butyrate (Sulfo-SMPB) or sulfosuccinimidyl 4-(N-maleimidomethyl) cyclohexane-1-carboxylate (Sulfo-SMCC).

An aqueous solution of sulfo-SMPB or sulfo-SMCC (Pierce Chemicals) was added to a solution of BSA or KLH in 0.02M sodium phosphate buffer (pH 7.0). The mixture was shaken at room temperature for 45 minutes and the activated carrier immediately applied to a Sephadex G-25 column equilibrated with 0.1M sodium phosphate buffer (pH 6.0) at 4° C.

The fractions of the first peak absorbance (280 nm) corresponding to activated carrier were combined in a round bottom flask to which was added a solution of peptide in 0.05M sodium phosphate buffer (pH 6.2). The mixture was thoroughly flushed with N₂ and incubated overnight at room temperature. The coupling efficiency was monitored using ³H-labeled peptide and by amino acid analysis of the conjugate.

Procedure 6

Detection of Antibodies to SARS-CoV by an Enzyme Linked Immunosorbent Assay (ELISA)

Each well of the microtiter plate is saturated with 100 μL of a solution (filtered 0.05M carbonate-bicarbonate buffer, pH 9.4±0.2) containing a peptide or mixture of peptides (5 μg/ml) and left overnight. Preferably, the inventors use an OsterBay Versafill dispensing system to fill the wells. The wells are emptied (preferably by aspiration) and washed twice with a washing buffer (NaCl, 0.15M; NaH₂PO₄, 0.060M; thimerosal, 0.01% and Tween 20, 0.05%; pH 7.4 (0.3 mL/well)). The wells are then saturated with 0.35 ml of washing buffer for 1 hour at 37° C. and washed once with the same buffer without Tween 20. After again drying for 1 hour at 37° C., the wells are ready for use. Serum samples to be analyzed are diluted with specimen buffer (containing sodium phosphate, 6 mM; NaCl, 0.15M and BSA, 2%, final pH is equal to 7.2). The wells are rinsed with washing buffer prior to the addition of the diluted serum sample (0.1 ml). These are left to incubate for 30 minutes at room temperature. The wells are then emptied, washed twice rapidly and then once for two minutes with washing buffer. The conjugate solution (peroxidase labeled affinity purified goat antibody to human IgG, 0.5 mg in 5 ml 50% glycerol) diluted with 1% w/v bovine serum albumin in a solution containing Tris, 0.05M; NaCl, 0.5M; Tween 20, 0.05%; thimerosal 0.01% (pH 7.2) is added to each well (0.1 ml) and incubated for 30 minutes at room temperature. The wells are then emptied and washed five times with the washing buffer. The substrate solution (3,3′,5,5′-tetramethyl-benzidine) (8 mg per ml of DMSO) is diluted with 100 volumes 0.1M citrate-acetate buffer (pH 5.6) containing 0.1% v/v of 30% H₂O₂ and added to each well (0.1 ml per well). After 10 minutes, the contents of each well are treated with 0.1 ml 2N H₂SO₄ and the optical density read at 450 nm. All determinations are done in duplicate.

### EXAMPLES

The following examples are illustrative of the wide range of applicability of the present invention and is not intended to limit its scope. Modifications and variations can be made therein without departing from the spirit and scope of the invention. Although any method and material similar or equivalent to those described herein can be used in the practice for testing of the present invention, the preferred methods and materials are described.

### Example 1

### Efficacy of the Peptides of the Invention for Detection of Anti-SARS-CoV Antibodies

The SARS-COV peptides corresponding to the sequences Seq ID nos:3, 17, 22, 23, 31, 34, 37, 101, 136 and 137 were chemically synthesized and used in a microplate EIA (Enzyme Immunoassay) for the detection of anti-SARS CoV IgG antibodies, according to the procedure described in the procedure 6.

To test the sensitivity of these peptides, a panel of 55 serum specimens collected from SARS-positive patients was prepared. The serological status of all these specimens was confirmed as SARS-positive by IFA (Indirect Fluorescence Assay). To test the specificity of the peptides, a panel of 22 serum specimens was prepared from a bank of sera collected from patients affected by other respiratory diseases in 2000 and 2001, at least two years before the reported appearance of SARS.

Results are shown in Table 1. For each peptide, a cutoff level was chosen based on the mean plus 3 standard deviations (Mean+3SD) of the results obtained with the 22 SARS-negative specimens. Any value beyond or below that cutoff value was classified as positive or negative, respectively. The 10 peptides showed a good specificity (95.5% or 100%) as no false-positive results was obtained with peptides Seq ID nos: 23, 37 and 137 while only 1 false-positive result was obtained with peptides corresponding to Seq ID nos: 3, 17, 22, 31, 34, 101, and 136. In terms of sensitivity, the peptides corresponding to Seq ID nos: 37, 136 and 137, respectively showed a significant reactivity with SARS-positive specimens (63.6%, 50.9% and 69.1% reactivity, respectively). Peptides of Seq ID nos:3, 22, 31, and 34 also showed some reactivity with 4, 5, 5 and 3 specimens detected out of 55, respectively.

In addition, when a mixture of peptides corresponding to Seq ID nos: 37, 136 and 137 was used for the testing, a sensitivity of 91.3% and a specificity of 100% was obtained (see Table 2, Mix 37-136-137). These results show that a combination of three synthetic peptides allows to improve the efficacy of detection of the assay, as the resultant sensitivity is better than that of the 3 individual peptides while the specificity remains the same (vs peptides Seq ID nos: 37 and 136) or becomes even superior to that of peptide of Seq ID no:137.

### Example 2

### Efficacy of the Peptides of the Invention for Detection of SARS-CoV Proteins

Rabbit were immunized with the peptides of Seq ID no:37 or Seq ID no:136 (both coupled with KLH). Serum was collected from these rabbits after 3 months and next tested against microplates coated with peptides of Seq ID nos: 37, 136 and 137, according to the procedure described in procedure 6. Addition of a goat anti-IgG-peroxidase conjugate revealed the presence of specific anti-peptide antibodies in all the antisera tested, but no significant response when the antiserum of Seq ID no 37 was tested with microplates coated with the peptides Seq ID nos 136 or 137 and when the antiserum of Seq ID no 136 was tested with microplates coated with the peptides Seq ID nos 37 or 137.

In the next set of experiments, one (1) microgram (μg) of recombinant nucleocapsid (N) protein (amino acids 1 to 49; Biodesign International, Saco, Me, USA) was added to the microwell, along with the rabbit antiserum. Results show that the added SARS N protein competed with the adsorbed peptide Seq ID no:136 for the antibody, as the signal decreased in presence of the protein, as shown in Table 2. A control experiment with the serum immunized with another peptide (Seq ID no:37) does not show the decrease in signal, indicating that this decrease is specific.

### Example-3

### Efficacy of the Peptides of the Invention the Detection of SARS-CoV Antigens

Microplates were coated with the recombinant N protein described in the Example 2 (1 μg/mL; 100 μL/well). The antisera described in the Example 2 were next added to the plates and IgG bound to the coated antigens were detected according to the procedure described in the procedure 6. Results obtained can be found in Table 3. They show that the antisera raised against SARS-CoV N peptides can be used to detect the N protein of SARS-CoV.

### REFERENCES

1. Drosten et al., 2003, Identification of a Novel Coronavirus in Patients with Severe Acute Respiratory Syndrome. N Engl J Med 348 (20): 1967-1976
2. Ksiazek et al., 2003, A Novel Coronavirus Associated with Severe Acute Respiratory Syndrome. N Engl J Med 348 (20): 1953-1966
3. Peiris et al., 2003, Coronavirus as a possible cause of severe acute respiratory syndrome. Lancet 361(9366):1319-25.
4. Ho et al., 2004, Antigenicity and receptor-binding ability of recombinant SARS coronavirus spike protein. Biochem Biophys Res Comm 313 (4): 938-947.
5. Wang et al.,2003, Assessment of immunoreactive synthetic peptides from the structural proteins of severe acute respiratory syndrome coronavirus. Clin Chem 49 (12): 1989-1996.

6. Wu et al., 2004, Early detection of antibodies against various structural proteins of the SARS-associated coronavirus in SARS patients. J Biomed Sc 11: 117-126.

TABLE 1
Sensitivity and Specificity of the SARS-CoV Peptides.

| | | OD 450 nm (- Blank) | | | | | |
|---|---|---|---|---|---|---|---|
| Sample ID | | Seq017 | Seq022 | Seq037 | Seq034 | Seq023 | Seq137 |
| SARS-Neg-01 | | .021 | .055 | .130 | .074 | .058 | .057 |
| SARS-Neg-02 | | .014 | .030 | .111 | .040 | .054 | .065 |
| SARS-Neg-03 | | .206 | .053 | .147 | .055 | .060 | .052 |
| SARS-Neg-04 | | .016 | .077 | .062 | .076 | .031 | .020 |
| SARS-Neg-05 | | .024 | .035 | .055 | .044 | .063 | .042 |
| SARS-Neg-06 | | .018 | .045 | .062 | .033 | .023 | .044 |
| SARS-Neg-07 | | .028 | .046 | .122 | .033 | .056 | .172 |
| SARS-Neg-08 | | .011 | .014 | .026 | .015 | .022 | .039 |
| SARS-Neg-09 | | .034 | .117 | .050 | .047 | .048 | .096 |
| SARS-Neg-10 | | .016 | .051 | .049 | .034 | .051 | .029 |
| SARS-Neg-11 | | .005 | .017 | .019 | .019 | .021 | .032 |
| SARS-Neg-12 | | .020 | .067 | .191 | .055 | .067 | .150 |
| SARS-Neg-13 | | .011 | .029 | .018 | .019 | .021 | .035 |
| SARS-Neg-14 | | .009 | .043 | .038 | .033 | .040 | .055 |
| SARS-Neg-15 | | .025 | .036 | .042 | .457 | .036 | .123 |
| SARS-Neg-16 | | .030 | .082 | .042 | .037 | .021 | .030 |
| SARS-Neg-17 | | .047 | .082 | .086 | .060 | .047 | .143 |
| SARS-Neg-18 | | .034 | .051 | .042 | .036 | .016 | .029 |
| SARS-Neg-19 | | .013 | .049 | .093 | .038 | .056 | .036 |
| SARS-Neg-20 | | .062 | .182 | .122 | .000 | .040 | .058 |
| SARS-Neg-21 | | .009 | .022 | .057 | .003 | .007 | .027 |
| SARS-Neg-22 | | .013 | .059 | .076 | .057 | .017 | .164 |
| SARS-Pos-01 | | .104 | .072 | .929 | .052 | .004 | .271 |
| SARS-Pos-02 | | .037 | .086 | .099 | .060 | .041 | .661 |
| SARS-Pos-03 | | .187 | .068 | .970 | .078 | .006 | .185 |
| SARS-Pos-04 | | .007 | .014 | .170 | .031 | .001 | .347 |
| SARS-Pos-05 | | .052 | .064 | .056 | .084 | .012 | 1.053 |
| SARS-Pos-06 | | .039 | .045 | 1.074 | .047 | .001 | .315 |
| SARS-Pos-07 | | .000 | .029 | 1.324 | .529 | .001 | .204 |
| SARS-Pos-08 | | .142 | .030 | 1.047 | .056 | .009 | .385 |
| SARS-Pos-09 | | .025 | .296 | .355 | .087 | .042 | 2.450 |
| SARS-Pos-10 | | .013 | .034 | .079 | .097 | .000 | 1.480 |
| SARS-Pos-11 | | .021 | .011 | .062 | .015 | .001 | .318 |
| SARS-Pos-12 | | .025 | .063 | .667 | .035 | .001 | .088 |
| SARS-Pos-13 | | .021 | .081 | .088 | .054 | .008 | .266 |
| SARS-Pos-14 | | .010 | .110 | .238 | .697 | .001 | 1.857 |
| SARS-Pos-15 | | .012 | .087 | .066 | .032 | .001 | 1.381 |
| SARS-Pos-16 | | .040 | .041 | 2.453 | .035 | .001 | .925 |
| SARS-Pos-17 | | .007 | .155 | .808 | .031 | .001 | 1.103 |
| SARS-Pos-18 | | .035 | 1.040 | .238 | .083 | .016 | 1.660 |
| SARS-Pos-19 | | .015 | .033 | 1.412 | .048 | .001 | .889 |
| SARS-Pos-20 | | .006 | .051 | 1.289 | .028 | .001 | .689 |
| SARS-Pos-21 | | .017 | .079 | .176 | .057 | .035 | .677 |
| SARS-Pos-22 | | .010 | .052 | 1.804 | .052 | .001 | 2.488 |
| SARS-Pos-23 | | .012 | .057 | .113 | .101 | .009 | 1.211 |
| SARS-Pos-24 | | .014 | .252 | .103 | .066 | .001 | .144 |
| SARS-Pos-25 | | .001 | .016 | .529 | .025 | .049 | .039 |
| SARS-Pos-26 | | .001 | .099 | .275 | .551 | .045 | .207 |
| SARS-Pos-27 | | .001 | .017 | .569 | .028 | .027 | .046 |
| SARS-Pos-28 | | .035 | .033 | .103 | .220 | .034 | 3.056 |
| SARS-Pos-29 | | .001 | .032 | .360 | .049 | .036 | .082 |
| SARS-Pos-30 | | .004 | .219 | .554 | .058 | .094 | .146 |
| SARS-Pos-31 | | .010 | .051 | .697 | .048 | .054 | .065 |
| SARS-Pos-32 | | .001 | .033 | .305 | .120 | .046 | 2.544 |
| SARS-Pos-33 | | .003 | .034 | .180 | .032 | .040 | 3.087 |
| SARS-Pos-34 | | .001 | .065 | .327 | .064 | .001 | .373 |
| SARS-Pos-35 | | .001 | .021 | .158 | .084 | .072 | .062 |
| SARS-Pos-36 | | .001 | .012 | .523 | .010 | .041 | .358 |
| SARS-Pos-37 | | .001 | .010 | .683 | .041 | .029 | .786 |
| SARS-Pos-38 | | .001 | .005 | .040 | .006 | .027 | .752 |
| SARS-Pos-39 | | .009 | .134 | 1.055 | .023 | .036 | 2.682 |
| SARS-Pos-40 | | .001 | .021 | .372 | .018 | .036 | .027 |
| SARS-Pos-41 | | .001 | .209 | .129 | .053 | .001 | .077 |
| SARS-Pos-42 | | .001 | .026 | .179 | .027 | .037 | .928 |
| SARS-Pos-43 | | .003 | .016 | 1.153 | .033 | .055 | 1.451 |
| SARS-Pos-44 | | .001 | .033 | .203 | .037 | .043 | .200 |
| SARS-Pos-45 | | .084 | .048 | 1.523 | .050 | .066 | .920 |
| SARS-Pos-46 | | .001 | .028 | .274 | .008 | .033 | .598 |
| SARS-Pos-47 | | .001 | .013 | .040 | .024 | .034 | .614 |
| SARS-Pos-48 | | .010 | .038 | .530 | .027 | .037 | .350 |
| SARS-Pos-49 | | .001 | .023 | .646 | .029 | .055 | .433 |
| SARS-Pos-50 | | .002 | .009 | .462 | .036 | .021 | .912 |
| SARS-Pos-51 | | .001 | .050 | .210 | .043 | .054 | .335 |
| SARS-Pos-52 | | .001 | .014 | 1.306 | .141 | .034 | .312 |
| SARS-Pos-53 | | .001 | .005 | 1.245 | .017 | .021 | .125 |
| | SARS-Pos-54 | .001 | .019 | .033 | .029 | .027 | .027 |
| | SARS-Pos-55 | .001 | .013 | 1.535 | .015 | .016 | .162 |
| Total | SARS-Neg | 22 | 22 | 22 | 22 | 22 | 22 |
| | SARS-Pos | 55 | 55 | 55 | 55 | 55 | 55 |
| Average OD | SARS-Neg | .030 | .056 | .075 | .058 | .039 | .068 |
| | SARS-Pos | .019 | .076 | .579 | .080 | .025 | .778 |
| SD OD | SARS-Neg | .041 | .037 | .046 | .091 | .018 | .049 |
| | SARS-Pos | .035 | .146 | .545 | .131 | .022 | .825 |

| | | | | | | |
|---|---|---|---|---|---|---|
| Cutoff (Mean Neg + 3SD) | .155 | .167 | .211 | .332 | .093 | .216 |
| Total TN | 21 | 21 | 22 | 21 | 22 | 22 |
| Total FP | 1 | 1 | 0 | 1 | 0 | 0 |
| Total TP | 1 | 5 | 35 | 3 | 1 | 38 |
| Total FN | 54 | 50 | 20 | 52 | 54 | 17 |
| Sens = | 1.8% | 9.1% | 63.6% | 5.5% | 1.8% | 69.1% |
| Spec = | 95.5% | 95.5% | 100% | 95.5% | 100% | 100% |

| | | OD 450 nm (- Blank) | | | | |
|---|---|---|---|---|---|---|
| Sample ID | | Seq003 | Seq136 | Seq101 | Seq031 | Mix 37-136-137 |
| SARS-Neg-01 | | .097 | .107 | .142 | .064 | .026 |
| SARS-Neg-02 | | .048 | .038 | .101 | .030 | .008 |
| SARS-Neg-03 | | .041 | .035 | .051 | .025 | |
| SARS-Neg-04 | | .016 | .016 | .017 | .011 | .013 |
| SARS-Neg-05 | | .026 | .027 | .034 | .022 | |
| SARS-Neg-06 | | .025 | .025 | .031 | .026 | .040 |
| SARS-Neg-07 | | .039 | .038 | .058 | .032 | |
| SARS-Neg-08 | | .016 | .021 | .023 | .015 | .007 |
| SARS-Neg-09 | | .022 | .017 | .028 | .017 | .022 |
| SARS-Neg-10 | | .024 | .026 | .029 | .017 | .006 |
| SARS-Neg-11 | | .013 | .020 | .016 | .008 | .007 |
| SARS-Neg-12 | | .026 | .042 | .047 | .038 | .044 |
| SARS-Neg-13 | | .015 | .016 | .017 | .009 | .015 |
| SARS-Neg-14 | | .014 | .011 | .025 | .209 | .003 |
| SARS-Neg-15 | | .019 | .020 | .027 | .012 | .009 |
| SARS-Neg-16 | | .008 | .016 | .028 | .007 | .012 |
| SARS-Neg-17 | | .033 | .035 | .045 | .028 | .028 |
| SARS-Neg-18 | | .013 | .014 | .018 | .012 | |
| SARS-Neg-19 | | .027 | .033 | .037 | .019 | |
| SARS-Neg-20 | | .050 | .045 | .086 | .073 | |
| SARS-Neg-21 | | .027 | .030 | .049 | .021 | |
| SARS-Neg-22 | | .046 | .046 | .081 | .038 | |
| SARS-Pos-01 | | .020 | .022 | .033 | .022 | .380 |
| SARS-Pos-02 | | .079 | .072 | .079 | .080 | |
| SARS-Pos-03 | | .026 | .030 | .039 | .024 | .407 |
| SARS-Pos-04 | | .012 | .292 | .020 | .010 | .280 |
| SARS-Pos-05 | | .031 | .032 | .042 | .024 | .049 |
| SARS-Pos-06 | | .024 | .030 | .028 | .028 | .190 |
| SARS-Pos-07 | | .025 | .515 | .050 | .020 | .581 |
| SARS-Pos-08 | | .031 | .033 | .038 | .025 | |
| SARS-Pos-09 | | .086 | .063 | .094 | .041 | 2.144 |
| SARS-Pos-10 | | .021 | .043 | .023 | .016 | .920 |
| SARS-Pos-11 | | .310 | .012 | .014 | .005 | .379 |
| SARS-Pos-12 | | .035 | .729 | .031 | .021 | .354 |
| SARS-Pos-13 | | .044 | .245 | .053 | .019 | .108 |
| SARS-Pos-14 | | .064 | .099 | .079 | .035 | 1.962 |
| SARS-Pos-15 | | .029 | .100 | .039 | .022 | |
| SARS-Pos-16 | | .026 | .038 | .045 | .082 | 2.027 |
| SARS-Pos-17 | | .029 | .073 | .033 | .014 | .923 |
| SARS-Pos-18 | | 1.820 | .102 | .080 | .078 | |
| SARS-Pos-19 | | .018 | .042 | .027 | .025 | 1.167 |
| SARS-Pos-20 | | .047 | .030 | .025 | .018 | .888 |
| SARS-Pos-21 | | .000 | .097 | .054 | .034 | .396 |
| SARS-Pos-22 | | .044 | 1.027 | .052 | .027 | 2.420 |
| SARS-Pos-23 | | .029 | 1.998 | .044 | .021 | |
| SARS-Pos-24 | | .034 | .028 | .045 | .341 | .082 |
| SARS-Pos-25 | | .011 | .527 | .032 | .011 | .039 |
| SARS-Pos-26 | | .022 | .062 | .073 | .032 | |
| SARS-Pos-27 | | .013 | .436 | .043 | .077 | |
| SARS-Pos-28 | | .045 | .335 | .035 | .021 | |
| SARS-Pos-29 | | .021 | .705 | .063 | .338 | |
| SARS-Pos-30 | | .206 | .416 | .273 | .103 | |
| SARS-Pos-31 | | .043 | .118 | .090 | .047 | .135 |
| SARS-Pos-32 | | .037 | .032 | .064 | .027 | |
| SARS-Pos-33 | | .028 | .080 | .054 | .021 | |
| SARS-Pos-34 | | .029 | .041 | .076 | .033 | |
| SARS-Pos-35 | | .078 | .232 | .082 | .047 | |
| SARS-Pos-36 | | .042 | .086 | .045 | .164 | |
| SARS-Pos-37 | | .044 | .059 | .058 | .034 | |
| SARS-Pos-38 | | .004 | .039 | .046 | .020 | |
| SARS-Pos-39 | | .043 | .250 | .044 | .019 | |
| SARS-Pos-40 | | .006 | .519 | .063 | .016 | |
| SARS-Pos-41 | | .177 | .123 | .245 | .107 | |
| SARS-Pos-42 | | .070 | .009 | .047 | .017 | |
| SARS-Pos-43 | | .016 | .148 | .047 | .184 | |
| SARS-Pos-44 | | .011 | .566 | .053 | .022 | |
| SARS-Pos-45 | | .057 | .189 | .116 | .042 | |
| SARS-Pos-46 | | .015 | .006 | .032 | .009 | |
| SARS-Pos-47 | | .021 | .047 | .068 | .050 | |
| SARS-Pos-48 | | .014 | .110 | .052 | .019 | |
| SARS-Pos-49 | | .024 | .060 | .062 | .024 | .389 |
| SARS-Pos-50 | | .034 | .225 | .021 | .098 | |
| SARS-Pos-51 | | .018 | .526 | .064 | .290 | |
| SARS-Pos-52 | | .024 | .023 | .052 | .020 | |
| SARS-Pos-53 | | .077 | .214 | .040 | .075 | .161 |
| | SARS-Pos-54 | .016 | .375 | .048 | .011 | |
| | SARS-Pos-55 | .021 | .024 | .050 | .013 | |
| Total | SARS-Neg | 22 | 22 | 22 | 22 | 14 |
| | SARS-Pos | 55 | 55 | 55 | 55 | 23 |
| Average OD | SARS-Neg | .029 | .031 | .045 | .033 | .017 |
| | SARS-Pos | .075 | .224 | .058 | .055 | .712 |
| SD OD | SARS-Neg | .019 | .020 | .032 | .043 | .013 |
| | SARS-Pos | .245 | .330 | .044 | .075 | .739 |

| | | | | | |
|---|---|---|---|---|---|
| Cutoff (Mean Neg + 3SD) | .088 | .091 | .141 | .161 | .056 |
| Total TN | 21 | 21 | 21 | 21 | 14 |
| Total FP | 1 | 1 | 1 | 1 | 0 |
| Total TP | 4 | 28 | 2 | 5 | 21 |
| Total FN | 51 | 27 | 53 | 50 | 2 |
| Sens = | 7.3% | 50.9% | 3.6% | 9.1% | 91.3% |
| Spec = | 95.5% | 95.5% | 95.5% | 95.5% | 100.0% |

TABLE 2

| Serum from rabbit | Recombinant N | |
|---|---|---|
| immunized with | protein (1-49) | |
| peptide of | added to the | Signal |
| Seq ID no; | serum (μg) | obtained |
| 37 | 0 | 1.446 |
| 37 | 1 | 1.237 |
| 136 | 0 | 1.555 |
| 136 | 1 | 0.774 |

TABLE 3

| Serum from rabbit immunized with | Signal obtained) |
|---|---|
| No serum (blank) | 0.022 |
| Seq ID no: 37 | 0.023 |
| Seq ID no: 37 | 0.025 |
| Seq ID no: 136 | 1.573 |
| Seq ID no: 136 | 1.598 |

1. An isolated peptide comprising an amino acid sequence selected from the group consisting of SEQ ID NOS: 1 to 4, 6 to 16, 18 to 22, 24 to 33, 35 to 41, 43 to 62, 64 to 73, 75 to 77, 79 to 81, 83 to 100, 102 to 105, 107 to 113, 115 to 118, 120 to 127 and 129 to 140 and analogues thereof.

2. An isolated peptide having the formula a-X-c-Z-b wherein: X and Z has an amino acid sequence independently selected from the group consisting of SEQ ID NOS: 1 to 4, 6 to 16, 18 to 22, 24 to 33, 35 to 41, 43 to 62, 64 to 73, 75 to 77, 79 to 81, 83 to 100, 102 to 105, 107 to 113, 115 to 118, 120 to 127 and 129 to 140 and analogues thereof, and wherein:
a is an amino terminus, one to eight amino acids or a substituent effective to facilitate coupling or to improve the immunogenic or antigenic activity of the peptide or to facilitate attachment to a support matrix;
b is a carboxy terminus, one to eight amino acids or a substituent effective to facilitate coupling or to improve the immunogenic or antigenic activity of the peptide or to facilitate attachment to the support matrix; and
c is a linker of one or two amino acids or a substituent effective to facilitate coupling of the two peptides in tandem or to improve the immunogenic or antigenic activity of the tandem peptide or to facilitate attachment to the support matrix.

3. The isolated peptide according to claim 1 or 2, wherein said amino acid sequence is selected from the group of amino acid sequences consisting of SEQ ID NOS: 3, 19, 22, 28, 31, 37, 136, 137, 138, 139 and 140 and analogues thereof.

4. The isolated peptide according to claim 1 or 2, wherein said amino acid sequence consists of SEQ ID NO: 3 or analogue thereof.

5. The isolated peptide according to claim 1 or 2, wherein said amino acid sequence consists of SEQ ID NO: 19 or analogue thereof.

6. The isolated peptide according to claim 1 or 2, wherein said amino acid sequence consists of SEQ ID NO: 22 or analogue thereof.

7. The isolated peptide according to claim 1 or 2, wherein said amino acid sequence consists of SEQ ID NO: 28 or analogue thereof.

8. The isolated peptide according to claim 1 or 2, wherein said amino acid sequence consists of SEQ ID NO: 31 or analogue thereof.

9. The isolated peptide according to claim 1 or 2, wherein said amino acid sequence consists of SEQ ID NO: 37 or analogue thereof.

10. The isolated peptide according to claim 1 or 2, wherein said amino acid sequence consists of SEQ ID NO: 136 or analogue thereof.

11. The isolated peptide according to claim 1 or 2, wherein said amino acid sequence consists of SEQ ID NO: 137 or analogue thereof.

12. The isolated peptide according to claim 1 or 2, wherein said amino acid sequence consists of SEQ ID NO: 138 or analogue thereof.

13. The isolated peptide according to claim 1 or 2, wherein said amino acid sequence consists of SEQ ID NO: 139 or analogue thereof.

14. The isolated peptide according to claim 1 or 2, wherein said amino acid sequence consists of SEQ ID NO: 140 or analogue thereof.

15. A mixture comprising at least two peptides or analogue thereof as defined in any one of claims 1 to 14.

16. An antibody that specifically binds to a peptide or analogue thereof as defined in any one of claims 1 to 14, or to a mixture as defined in claim 15.

17. The antibody of claim 16, characterised in that said antibody consists of a monoclonal or polyclonal antibody.

18. A mixture comprising at least two antibodies as defined in claims 16 or 17.

19. An in vitro diagnostic method for the detection of the presence or absence of antibodies indicative of SARS-CoV, which bind with a peptide or analogue thereof according to any one of claims 1 to 14 to form an immune complex, comprising the steps of:
a) contacting the peptide or analogue thereof according to any one of claims 1 to 14 with a biological sample for a time and under conditions sufficient to form an immune complex; and
b) detecting the presence or absence of the immune complex formed in a).

20. A diagnostic kit for the detection of the presence or absence of antibodies indicative of SARS-CoV, comprising:
a peptide or analogue thereof according to any one of claims 1 to 14 and
a reagent to detect a peptide-antibody immune complex:
wherein said peptide or analogue thereof, reagent, biological reference sample and comparison sample are present in an amount sufficient to perform said detection.

21. The kit of claim 19, further comprising-a reagent to detect a peptide-antibody immune complex and/or a biological reference sample lacking antibodies that immunologically bind with said peptide or analogue thereof, wherein said biological reference sample and comparison sample are present in an amount sufficient to perform said detection.

22. An in vitro diagnostic method for the detection of the presence or absence of peptides or proteins indicative of SARS-CoV, which bind with an antibody according to claim 16 or 17 to form an immune complex, comprising the steps of:
a) contacting the antibody according to claim 16 or 17 with a biological sample for a time and under conditions sufficient to form an immune complex; and
b) detecting the presence or absence of the immune complex formed in a).

23. A diagnostic kit for the detection of the presence or absence of peptides or proteins indicative of SARS-COV, comprising:
an antibody according to claim 16 or 17 and
a reagent to detect a peptide-antibody immune complex;
wherein said antibody, reagent, biological reference sample and comparison sample are present in an amount sufficient to perform said detection.

24. The kit of claim 23, further comprising a biological reference sample lacking peptides that immunologically bind with said antibody and/or a comparison sample comprising peptides which can specifically bind to said antibody, wherein said biological reference sample and comparison sample are present in an amount sufficient to perform said detection.
